(19) United States
(12) Patent Application Publication
(10) Pub. No.: US 2026/0157733 A1
(43) Pub. Date: Jun. 11, 2026

(54) SYSTEM AND METHOD FOR QUALITY CHECKING REPROCESSED INTRAVASCULAR ULTRASOUND CATHETERS

(71) Applicants: Glenn Meyer, Windermere, FL (US); Seth Masek, Dover, FL (US); Michael Garrett, Cincinnatti, OH (US)

(72) Inventors: Glenn Meyer, Windermere, FL (US); Seth Masek, Dover, FL (US); Michael Garrett, Cincinnatti, OH (US)

(21) Appl. No.: 18/974,010

(22) Filed: Dec. 9, 2024

(51) Int. Cl. A61B 8/00

(52) U.S. Cl. CPC A61B 8/58; A61B 8/445

(57) ABSTRACT

The invention is a system and method for quality-checking reprocessed IVUS catheters for electrical discontinuities during simulated use involving distal-end deflection.

The application U.S. Ser. No. 18/941530, with filing date 11 Aug. 2024 is incorporated by reference herein.

### TECHNICAL FIELD

The invention is a system and method for electrically testing reprocessed intravascular ultrasound (IVUS) catheters for discontinuities during simulated-use deflection.

### BACKGROUND OF INVENTION

An increasingly common means for determining internal vascular conditions relies on IVUS catheters manipulated axially within a vein's lumen.

Solid-state IVUS devices incorporate a ring of multiple ultrasound transducers that both emit and receive ultrasound beams directed to, and reflected from, interior vascular wall surfaces.

These IVUS catheters have an electrical cable whose conductors are soldered to connector pins within the IVUS device. Any change in conductivity when an IVUS is being manipulated can cause inaccuracies in displayed imagery or loss of image altogether.

New, pre-first-use IVUS devices have been quality checked by their original equipment manufacturers (OEMs). During first-use, there is a finite possibility of changes in conductivity and resulting image degradation or loss. Reprocessed IVUS catheters that have already been used at least once have a higher possibility of changes in conductivity and resulting image degradation or loss.

Therefore, a quality check of reprocessed IVUS catheters could markedly reduce those possibilities and improve the reliance on such devices.

### BRIEF DESCRIPTION OF INVENTION

The invention is a test system used in conjunction with an IVUS catheter, an OEM imaging system and patient interface module (PIM).

It was found after extensive testing and evaluation that the likelihood of quality-check detection of reprocessed catheters with highest likelihood of discontinuity problems could be determined by simulating the deflection of the distal portion of the IVUS catheter, during simulated-use operation, while monitoring and sampling the current flow from the catheter's pin 9 to its ground reference.

After testing of all models of IVUS solid-state catheters having multiple transducers, the models PV.014, PV.018 and PV.035 all exhibited a correlation between higher problem likelihood and deviation of current flow from their pin 9 to the ground reference during simulated-use and distal deflection.

A range of current values was found within which the likelihood of problematic operation substantially decreases for reprocessed IVUS catheters among these three IVUS models.

The invention is a tester subsystem that connects between the PIM and catheter and which samples the current values from pin 9 to ground reference with a predetermined sampling rate. Any deviation outside the predetermined range of current values is considered a test failure and eliminated. Those that have no deviating values are considered as having passed the test and are made available for reprocessed catheter service.

### BRIEF DESCRIPTION OF DRAWINGS

FIG. 1 depicts an IVUS catheter consistent with the design of the PV.14, PV.18 and PV.35 family of devices.

FIG. 2 depicts a CT scan of a failed IVUS showing the point of failure.

FIG. 3 depicts the pin connectors that are integral to the IVUS catheters and to which the electrical cable's conductors are soldered.

FIG. 4 depicts an embodiment of the invention used in conjunction with an OEM imaging system, its PIM, and an IVUS catheter.

FIG. 5 depicts an embodiment's functional blocks and their interrelationships.

FIG. 6 depicts an embodiment of the test subsystem and its interface elements.

FIG. 7 depicts the range of all current values as captured during testing for all devices within the PV.14, PV.18, and PV.35 IVUS device families. The histogram sorts all test samples into electrical current intervals. The y-axis shows the occurrence rate of discontinuities during simulated-use operation per electrical current interval.

FIG. 8 depicts all the sampled currents from pin 9 to ground for a particular device wherein deviations outside the predetermined range are clearly captured.

FIG. 9 is a method-step flow for an embodiment of the invention.

### DETAILED DESCRIPTION OF INVENTION

The invention is a testing subsystem used in conjunction with an OEM imaging system, its PIM, and an IVUS catheter.

It was found that IVUS catheters which exhibited normal operational behavior could, under use, where the distal end is deflected, experience discontinuities in electrical flow that can result in image degradation or image loss. Either condition would not be acceptable during actual use. However, static testing of an IVUS device would not show any proclivity toward discontinuities under deflection.

Consequently, a test system and method was invented that could quickly and reliably weed out IVUS catheters at risk of discontinuities under deflection.

After extensive research and experimentation, it was found that if one monitored the current flow between an IVUS catheter's pin 9 and its ground reference, it was possible to quality-check an IVUS catheter to determine its likelihood of consistent quality under normal use conditions.

While the IVUS catheter was in operation mode, and the current flow from pin 9 to ground was being sampled at a high rate (500-1000 Hz), catheters that continued to perform properly were frequently found to have pin 9 current values that fell within a specific range of values. This is called “a predetermined range of current values” for the sake of lexicon. Those catheters whose pin 9 current readings deviated from the predetermined range of current values were found to have unacceptable likelihoods of quality degradation under normal use and deflection.

Based on these findings, the test system essentially sits between the PIM and IVUS catheter and samples and captures pin 9 current values which are conveyed to a computing subsystem.

The values conveyed are compared, by the computing subsystem, to the predetermined range of current values.

If there are no readings outside that predetermined range, despite typical distal-end deflection, the IVUS device under test is considered having passed the quality check and is considered eligible for use. Any device whose values fall outside the predetermined range is considered having failed the quality check and is eliminated from further processing and use.

The following drawings and descriptions are for an embodiment of the invention and are included for further elucidation but should not be read as limiting the scope of claims.

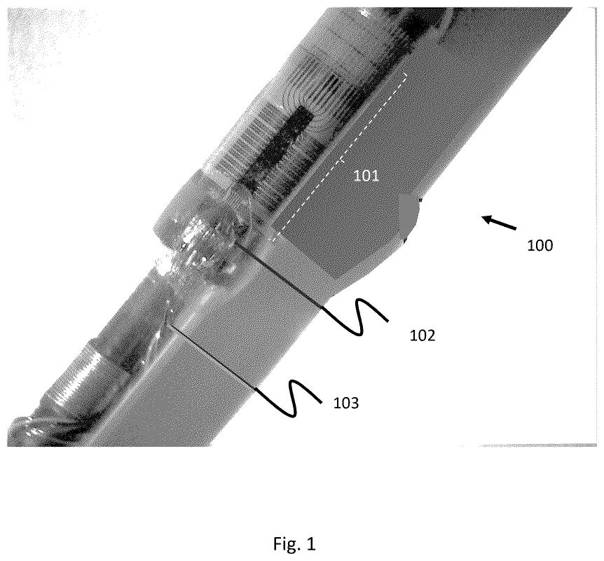

FIG. 1 shows an IVUS catheter (100) with its transducers (101), its electrical interface to its cable (102) and an end portion of its cable (103). This drawing would be the same for IVUS family members of PV.14, PV.18 and PV.35 model devices.

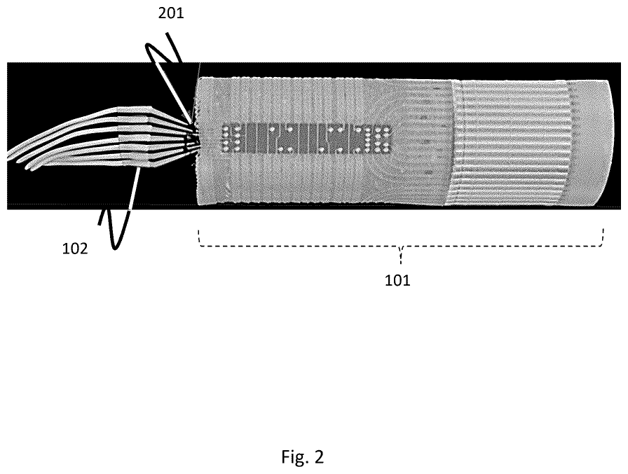

FIG. 2 is a CT scan of a failed IVUS catheter showing the transducers (101), and the electrical conductors (102) where the point of failure (201) is visible.

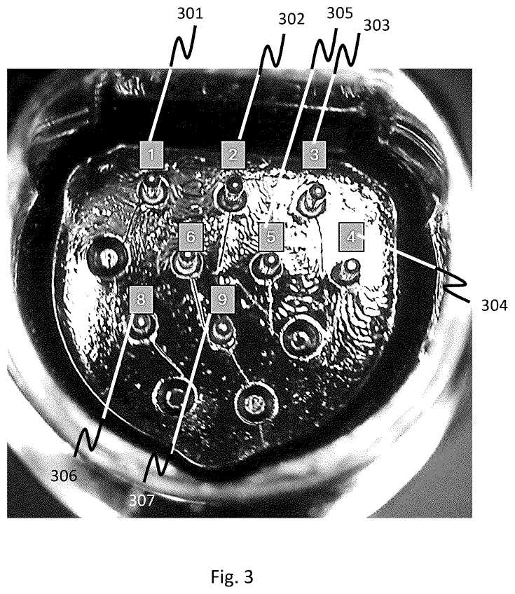

FIG. 3 shows a cross-section view of the pin out wherein the conductors and pins are interfaced and soldered. During operation of the catheter, the OEM imaging system and its PIM are providing input and output signals. Pins 1-4 (301, 302, 303, 304) and 8 (306) are all related to signals that could cause a loss of image. Pins 5 and 6 (305 ) can cause image degradation but no loss of image. Thus, measuring current through those pins 1-4 and 8 could find anomalies related to loss of image. However, by sampling just the current at pin 9 (307), there was equal correlation to be found and a simpler sampling routine.

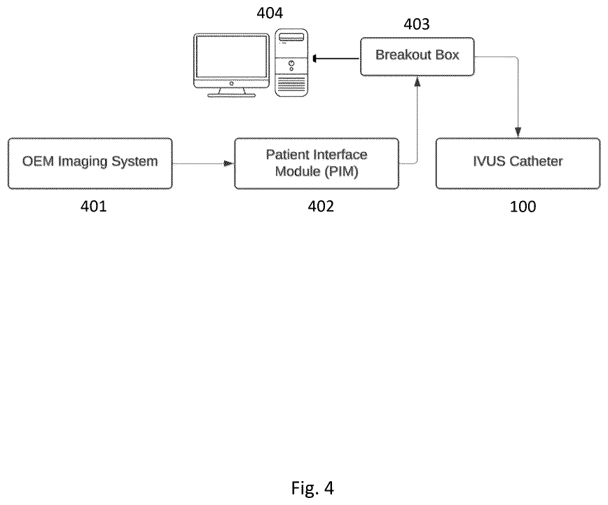

In FIG. 4, the testing subsystem (403) is connected to a computing subsystem (404), the PIM (402) and the IVUS catheter.

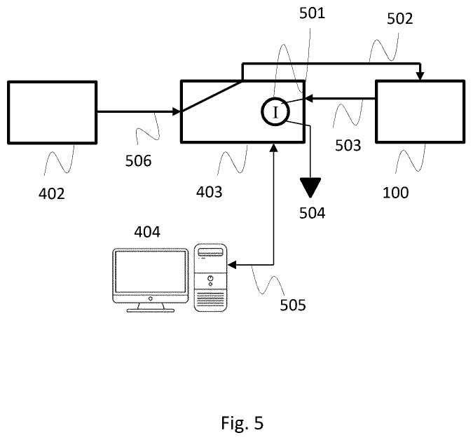

In FIG. 5, the PIM (402) provides operational signals through the interface cable (506) between test subsystem (403) and 402. The operational signals are conveyed to the IVUS catheter (100) via its interface cable (502). And, the test subsystem (403) is interfaced to its computing subsystem (404) via its interface cable (505).

When so configured and connected, the test subsystem (403) and its microcontroller are operative to measure the catheter's pin 9 current flow via path 503 to its ground reference, 504.

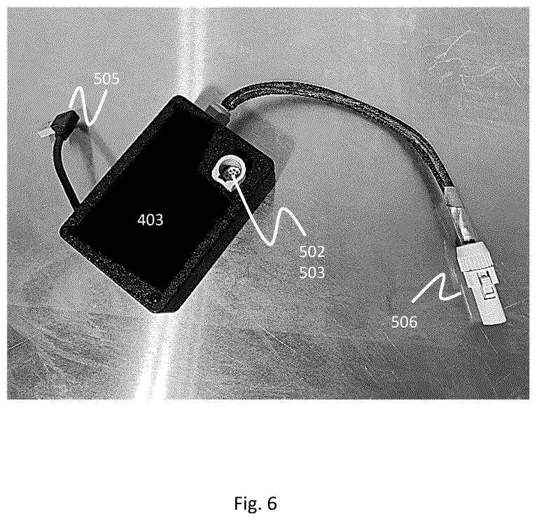

An embodiment of the test subsystem (403) is shown in FIG. 6 wherein the interface to the computing subsystem (505) that to the IVUS catheter (502 and 503), and the interface to the PIM (506) are shown.

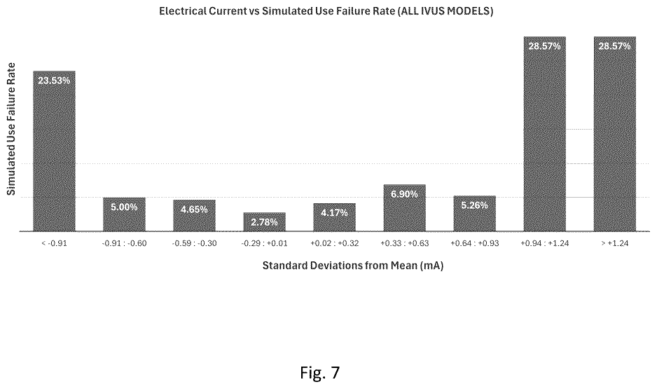

FIG. 7 shows a full range of current values sampled and recorded for a large number of IVUS devices under test. Devices whose readings fell below −0.91 and above +0.93 standard deviations (mA) from the sample mean were found to have a significantly higher likelihood of problems under normal use with typical distal-end deflection. Those within the range of −0.91 and +0.93 standard deviations had a much higher likelihood of proper operation under normal use with typical distal-end deflection. Thus, in this embodiment, the range −0.91 to +0.93 standard deviations was the predetermined range of current values used to determine test passing or failure.

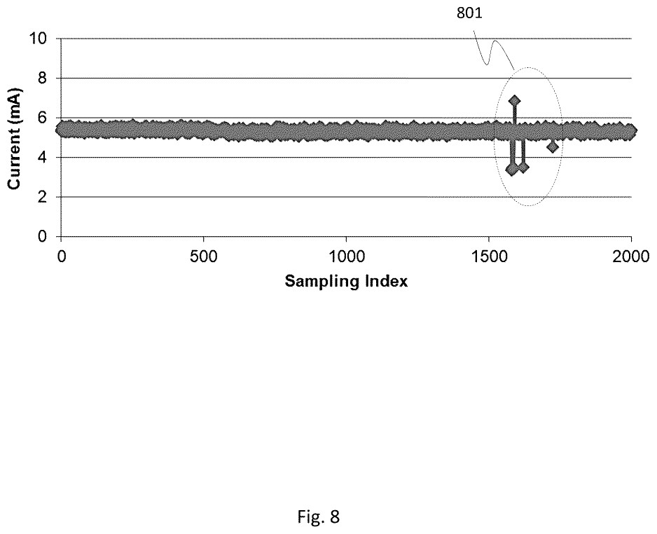

FIG. 8 shows an example of an IVUS device under test that showed deviant readings (801) around sample 1600 and on. This would be a device that would be considered as having failed the quality-check test and would be removed from further processing.

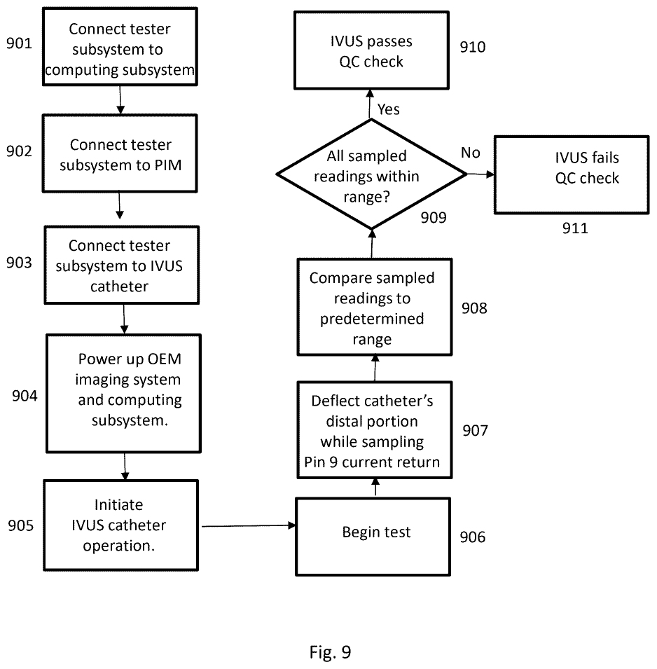

This embodiment's method of use, shown in FIG. 9, is to first connect the tester to the computing subsystem (901), then connect it to the PIM (902), then connect it to the IVUS catheter. After all connections are made, the OEM imaging system and computing subsystem are powered up providing power to the catheter and test subsystem. Next, the IVUS catheter's operation is initiated via the OEM imaging system (905) and the test begins (906) initiated by a program in the computing subsystem. While the test subsystem is sampling the current flow between pin 9 and reference ground, the distal-portion of the catheter is deflected as might be the case in actual use (907). The current-value samples are compared by the computing subsystem to readings across a predetermined range (908). Where all sampled readings fall within that range (909), the computing subsystem indicates the IVUS has passed the quality-check (QC) test (910). Where one or more readings are outside the predetermined range, the computing subsystem indicates the IVUS has failed the QC test (911).

The sampling rate between 500 and 1000 Hz is not critical, and the overall test time is not critical so long as normally expected distal-portion deflection is occurring during the sampling. For testing other IVUS devices besides those of the PV.14, PV.18 and PV.35 families, one could experiment and come up with a predetermined range of values and use a pin analogous to pin 9 for sampling the current values.

What is claimed is:

1. A system for quality-checking reprocessed solid-state intravascular ultrasound (IVUS) catheters having multiple ultrasound transducers and a connector including a pin 9 and a ground reference, comprising:
a testing subsystem configured to be connected between an imaging system provided by an original equipment manufacturer (OEM) of the IVUS catheter, a patient interface module (PIM), and the IVUS catheter;
a computing subsystem;
wherein, when the IVUS catheter is operated by the imaging system via the PIM and a distal portion of the IVUS catheter is mechanically deflected to simulate normal clinical use, the testing subsystem comprises:
a current measurement device coupled between the connector pin 9 of the IVUS catheter and the ground reference and configured to generate current-measurement samples at a predetermined sampling rate; and
a microcontroller subsystem configured, under program control, to receive the current-measurement samples and convey the current-measurement samples to the computing subsystem; and
wherein the computing subsystem is configured, under control of at least one program, to compare the current-measurement samples to a predetermined range of pin-9 current values determined from testing a population of IVUS catheters of a same model family under simulated-use distal deflection, and to indicate that the reprocessed IVUS catheter has passed a quality-check test when no current-measurement samples fall outside the predetermined range and to indicate that the reprocessed IVUS catheter has failed the quality-check test when at least one current-measurement sample falls outside the predetermined range.

2. A system as in claim 1 wherein:
the testing subsystem comprises:
the microcontroller subsystem;
the at least one program;
the current measurement device;
an interface connector for interface of the testing subsystem with the IVUS catheter;
an interface cable for interface of the testing subsystem with the imaging system; and
an interface cable for interface of the testing subsystem with the computing subsystem.

3. A system as in claim 2 wherein:
the microcontroller subsystem comprises:
a central processing unit subsystem;
a program memory subsystem;
a data memory subsystem;
an interface subsystem operative to exchange signals with the imaging system, the PIM, the IVUS catheter, and the computing subsystem; and
an analog-to-digital conversion subsystem.

4. A system as in claim 1 wherein:
the computing subsystem comprises:
a central processing unit subsystem;
a data memory subsystem;
a program memory subsystem;
at least one program; and
an interface operative to interface the computing subsystem with the testing subsystem and to receive the current-measurement samples therefrom.

5. A system as in claim 1 wherein:
the predetermined sampling rate is between 500 Hz and 1000 Hz and the predetermined range of pin-9 current values corresponds to a range between −0.91 and 0.93 standard deviations from a sample mean of pin-9 current values obtained from the population of IVUS catheters of the same model family.

6. A system as in claim wherein the indication that the reprocessed IVUS catheter has passed the quality-check test comprises generating a signal to authorize the reprocessed IVUS catheter for further clinical use, and wherein the indication that the reprocessed IVUS catheter has failed the quality-check test comprises generating a signal to remove the reprocessed IVUS catheter from further processing and use.
